SEQUENCE LISTING (16 sequences)

SEQ ID NO: 1 - protein, 8 aa, Artificial Sequence
  Derivative of Jelleine-1 of Apis Mellifera
PFKLKLHL

SEQ ID NO: 2 - protein, 8 aa, Artificial Sequence
  Derivative of Jelleine-1 of Apis Mellifera
  MISC_FEATURE (3)..(3): X=Orn
PFXLSLKL

SEQ ID NO: 3 - protein, 8 aa, Artificial Sequence
  Derivative of Jelleine-1 of Apis Mellifera
  MISC_FEATURE (3)..(3): X=Dab
PFXLKLKL

SEQ ID NO: 4 - protein, 8 aa, Artificial Sequence
  Derivative of Jelleine-1 of Apis Mellifera
PFRLSLHL

SEQ ID NO: 5 - protein, 8 aa, Artificial Sequence
  Derivative of Jelleine-1 of Apis Mellifera
PFRLRLHL

SEQ ID NO: 6 - protein, 8 aa, Artificial Sequence
  Derivative of Jelleine-1 of Apis Mellifera
PFRLSLRL

SEQ ID NO: 7 - protein, 8 aa, Artificial Sequence
  Derivative of Jelleine-1 of Apis Mellifera
PFRLRLRL

SEQ ID NO: 8 - protein, 8 aa, Artificial Sequence
  Derivative of Jelleine-1 of Apis Mellifera
PWKLSLHL

SEQ ID NO: 9 - protein, 8 aa, Artificial Sequence
  Derivative of Jelleine-1 of Apis Mellifera
  MISC_FEATURE (3)..(3): X=Orn
  MISC_FEATURE (5)..(5): X=Orn
PWXLXLHL

SEQ ID NO: 10 - protein, 8 aa, Artificial Sequence
  Derivative of Jelleine-1 of Apis Mellifera
  MISC_FEATURE (3)..(3): X=Dab
  MISC_FEATURE (7)..(7): X=Dab
PWXLSLXL

SEQ ID NO: 11 - protein, 8 aa, Artificial Sequence
  Derivative of Jelleine-1 of Apis Mellifera
  MISC_FEATURE (3)..(3): X=Dap
  MISC_FEATURE (5)..(5): X=Dap
  MISC_FEATURE (7)..(7): X=Dap
PWXLXLXL

SEQ ID NO: 12 - protein, 8 aa, Artificial Sequence
  Derivative of Jelleine-1 of Apis Mellifera
PWRLSLHL

SEQ ID NO: 13 - protein, 8 aa, Artificial Sequence
  Derivative of Jelleine-1 of Apis Mellifera
PWRLRLHL

SEQ ID NO: 14 - protein, 8 aa, Artificial Sequence
  Derivative of Jelleine-1 of Apis Mellifera
PWRLSLRL

SEQ ID NO: 15 - protein, 8 aa, Artificial Sequence
  Derivative of Jelleine-1 of Apis Mellifera
PWRLRLRL

SEQ ID NO: 16 - protein, 8 aa, Artificial Sequence
  Natural Jelleine-1 of Apis Mellifera
PFKLSLHL

(19) United States
(12) Patent Application Publication
(10) Pub. No.: US 2025/0345492 A1
(43) Pub. Date: Nov. 13, 2025

(54) AN ANTIBACTERIAL POLYPEPTIDE COMPOUND, A MEDICAL DEVICE, HYDROGEL AND USE THEREOF

(71) Applicant: GuangZhou Towe Biotech Co., Ltd., Guangzhou (CN)

(72) Inventor: Yue LI, Guangzhou (CN)

(21) Appl. No.: 18/270,395

(22) PCT Filed: Dec. 17, 2021
(86) PCT No.: PCT/CN2021/139094

§ 371 Date: Jun. 29, 2023

(30) Foreign Application Priority Data
Dec. 30, 2020 (CN) 202011613292.8
Mar. 4, 2021 (CN) 202110240501.7
Apr. 15, 2021 (CN) 202110407232.9

(51) Int. Cl. A61L 29/14; A61K 9/06; A61K 38/00; A61L 29/08; A61L 29/16; C07K 7/06

(52) U.S. Cl. CPC A61L 29/145; A61K 9/06; A61L 29/08; A61L 29/16; C07K 7/06; A61K 38/00

(57) ABSTRACT

Disclosed by the present invention are an antibacterial polypeptide compound, a medical device, hydrogel and a use thereof. The antibacterial polypeptide compound has high antibacterial activity, high enzymatic hydrolysis stability, high bioavailability and low cytotoxicity. The hydrogel of the present invention does not adhere to wounds, has antibacterial activity and hemostatic properties, and can be used for loading and controlled-release of a drug, for example, an anti-inflammatory drug, an epidermal growth factor, a vascular growth factor or the like may be loaded; moreover, the hydrogel may accelerate healing of the wounds and reduce the formation of scar tissue fibers. In addition, the preparation method of the hydrogel of the present invention involves few processing steps and few types of raw materials and is convenient to perform.

### CROSS-REFERENCE

The present application is a nationalization of PCT Application No. PCT/CN2021/139094 filed on Dec. 17, 2021, which claims priority to CN application Ser. No. 20/211,0407232.9 filed on Apr. 15, 2021, priority to CN application Ser. No. 20/211,0240501.7 filed on Mar. 4, 2021, and priority to CN application Ser. No. 20/201,1613292.8 filed on Dec. 30, 2020, which applications are incorporated herein by specific reference in their entirety.

### SEQUENCE LISTING

This application contains a Sequence Listing which has been submitted in ASCII format and is hereby incorporated by reference in its entirety. The ASCII copy, is named L1211-10055US01_Sequence_Listing created on Jan. 16, 2024 and is 4,263 bytes in size.

### TECHNICAL FIELD OF THE INVENTION

The present invention belongs to the field of biochemical technology, and relates to an antibacterial polypeptide compound, particular to an antibacterial polypeptide compound, hydrogel and a use thereof for antibiosis and hemostasis, and a medical device suitable for the antibacterial polypeptide compound or the hydrogel.

### BACKGROUND OF THE INVENTION

With the overuse and abuse of antibiotics in medical, agricultural and food industries, the dramatically evolved resistance of bacteria to the antibiotics greatly compromises the efficacy of the antibiotics of prior art and severely threatens global public health and safety. There are about 700,000 deaths worldwide caused by drug-resistant bacterial infections every year. By 2050, it is estimated that 10 million people will die from such infections every year, and related economic losses will exceed trillions of dollars. WHO has called the antibacterial resistance “one of the biggest threats to global health, food security and development today” (Chinese Journal of Biotechnology, 2018, 34 (8): 1346-1360). In order to cope with the challenge of antibacterial resistance to human health and safety, development of new antibacterial drugs has become one of the most urgent medical issues.

In view of the advantages of polypeptide molecules compared with small-molecule drugs, such as larger binding area, stronger targeting, safer, less side effects and less possibility of causing severe immune response. The antibacterial polypeptide molecule has become a popular field in the research and development of emerging antibacterial drugs. However, natural antibacterial polypeptides are often limited in clinical applicability due to low antibacterial activity and unknown systemic toxicity. The changes in amino acid construct and side chain and chemical modification of the antibacterial polypeptide chains often produce unpredictable influences on the antibacterial activity and toxicity. Therefore, amino acid modification and substitution in antibacterial peptide compounds is also an important strategy to obtain antibacterial polypeptide derivatives with high efficiency and low toxicity.

In recent years, hydrogels have been regarded as biological materials that have promising potential for antibiosis, hemostasis, wound healing and anti-adhesion. Generally, the raw materials for preparing hydrogels are mainly divided into two categories, one is synthetic polymer, and the other is natural biological materials, such as polysaccharide, protein and polypeptide. Wherein, the polypeptide is easily hydrolyzed into amino acids by protease in vivo and has no adverse effects on the body, so it has less side effects than the polymeric hydrogel of prior art.

Antibacterial polypeptide J-1 (Jelleine-1) is first natural antibacterial polypeptide obtained from royal jelly of Apismellifera, which has broad-spectrum antibacterial and antifungal activities (Peptides, 2004, 25:919-928).

However, the antibacterial polypeptide J-1 have the same defects as other natural antibacterial polypeptides, including low antibacterial activity and low bioavailability, which severely restricts their use (Peptides, 2019, 112:56-66). In order to improve the biological activity and clinical applicability of the antibacterial polypeptide J-1, the present invention designs and synthesizes a series of antibacterial polypeptide J-1 derivatives based thereon. According to researches, we found that these derivatives exhibit better antibacterial activity, lower cytotoxicity and better enzymatic hydrolysis stability than the antibacterial polypeptide J-1, and all of them are prepared into hydrogels by means of a simple method, so they are promising to be developed into biological dressings with antibiosis, hemostasis, wound-healing promoting and other functions.

### SUMMARY OF THE INVENTION

The main objective of the present invention is to provide an antibacterial polypeptide compound, which has high antibacterial activity, high enzymatic hydrolysis stability, high bioavailability and low cytotoxicity.

In order to achieve the above-mentioned objective, the present invention provides an antibacterial polypeptide compound, which comprises a parent peptide represented by the following amino acid sequence:

Pro-Xaa2-Xaa3-Leu-Xaa5-Leu-Xaa7-Leu-NH₂

wherein, Xaa2=Phe, homo-Phe or Trp;
Xaa3=Lys, Aib, Orn, Dab, Dap or Arg;
Xaa5 =Ser, Lys, Orn, Dab, Dap or Arg;
Xaa7 =His, Lys, Orn, Dab, Dap or Arg;
and if Xaa2 =Phe, Xaa3 =Lys and Xaa5 =Ser, Xaa7/His.

If Xaa2=Phe, Xaa3=Lys, Xaa5=Ser and Xaa7=His, the amino acid sequence represented is the amino acid sequence of the natural antibacterial polypeptide J-1, which is outside the protection scope of the present invention.

If Xaa2=Phe, Xaa3=Lys, Xaa5=Lys and Xaa7=His, the antibacterial polypeptide compound is labeled as compound 1 (related to SEQ ID NO:1):

Pro-Phe-Lys-Leu-Lys-Leu-His-Leu-NH₂
PFKLKLHL-NH₂.

If Xaa2=Phe, Xaa3=Orn, Xaa5=Ser and Xaa7=Lys, the antibacterial polypeptide compound is labeled as compound 2 (related to SEQ ID NO:2):

Pro-Phe-Orn-Leu-Ser-Leu-Lys-Leu-NH₂
PF-Orn-LSLKL-NH₂.

If Xaa2=Phe, Xaa3=Dab, Xaa5=Lys and Xaa7=Lys, the antibacterial polypeptide compound is labeled as compound 3 (related to SEQ ID NO:3):

Pro-Phe-Dab-Leu-Lys-Leu-Lys-Leu-NH₂
PF-Dab-LKLKL-NH₂.

If Xaa2=Phe, Xaa3=Arg, Xaa5=Ser and Xaa7=His, the antibacterial polypeptide compound is labeled as compound 4 (related to SEQ ID NO:4):

Pro-Phe-Arg-Leu-Ser-Leu-His-Leu-NH₂
PFRLSLHL-NH₂.

If Xaa2=Phe, Xaa3=Arg, Xaa5=Arg and Xaa7=His, the antibacterial polypeptide compound is labeled as compound 5 (related to SEQ ID NO:5):

Pro-Phe-Arg-Leu-Arg-Leu-His-Leu-NH₂
PFRLRLHL-NH₂.

If Xaa2=Phe, Xaa3=Arg, Xaa5=Ser and Xaa7=Arg, the antibacterial polypeptide compound is labeled as compound 6 (related to SEQ ID NO:6):

Pro-Phe-Arg-Leu-Ser-Leu-Arg-Leu-NH₂
PFRLSLRL-NH₂.

If Xaa2=Phe, Xaa3=Arg, Xaa5=Arg and Xaa7=Arg, the antibacterial polypeptide compound is labeled as compound 7 (related to SEQ ID NO:7):

Pro-Phe-Arg-Leu-Arg-Leu-Arg-Leu-NH₂
PFRLRLRL-NH₂.

If Xaa2=Trp, Xaa3=Lys, Xaa5=Ser and Xaa7=His, the antibacterial polypeptide compound is labeled as compound 8 (related to SEQ ID NO:8):

Pro-Trp-Lys-Leu-Ser-Leu-His-Leu-NH₂
PWKLSLHL-NH₂.

If Xaa2=Trp, Xaa3=Orn, Xaa5=Orn and Xaa7=His, the antibacterial polypeptide compound is labeled as compound 9 (related to SEQ ID NO:9):

Pro-Trp-Orn-Leu-Orn-Leu-His-Leu-NH₂
PW-Orn-L-Orn-LHL-NH₂.

If Xaa2=Trp, Xaa3=Dab, Xaa5=Ser and Xaa7=Dab, the antibacterial polypeptide compound is labeled as compound 10 (related to SEQ ID NO:10):

Pro-Trp-Dab-Leu-Ser-Leu-Dab-Leu-NH₂
PW-Dab-LSL-Dab-L-NH₂.

If Xaa2=Trp, Xaa3=Dap, Xaa5=Dap and Xaa7=Dap, the antibacterial polypeptide compound is labeled as compound 11 (related to SEQ ID NO:11):

Pro-Trp-Dap-Leu-Dap-Leu-Dap-Leu-NH₂
PW-Dap-L-Dap-L-Dap-L-NH₂.

If Xaa2=Trp, Xaa3=Arg, Xaa5=Ser and Xaa7=His, the antibacterial polypeptide compound is labeled as compound 12 (related to SEQ ID NO:12):

Pro-Trp-Arg-Leu-Ser-Leu-His-Leu-NH₂
PWRLSLHL-NH₂.

If Xaa2=Trp, Xaa3=Arg, Xaa5=Arg and Xaa7=His, the antibacterial polypeptide compound is labeled as compound 13 (related to SEQ ID NO:13):

Pro-Trp-Arg-Leu-Arg-Leu-His-Leu-NH₂
PWRLRLHL-NH₂.

If Xaa2=Trp, Xaa3=Arg, Xaa5=Ser and Xaa7=Arg, the antibacterial polypeptide compound is labeled as compound 14 (related to SEQ ID NO:14):

Pro-Trp-Arg-Leu-Ser-Leu-Arg-Leu-NH₂
PWRLSLHL-NH₂.

If Xaa2=Trp, Xaa3=Arg, Xaa5=Arg and Xaa7=Arg, the antibacterial polypeptide compound is labeled as compound 15 (related to SEQ ID NO:15):

Pro-Trp-Arg-Leu-Arg-Leu-Arg-Leu-NH₂
PWRLRLRL-NH₂.

The amino acid sequences of the above-mentioned compounds 1-15 and the natural antibacterial polypeptide J-1 are shown in Table 1.

TABLE 1
Amino Acid Sequences of the Compounds 1-15 and the Natural
Antibacterial Polypeptide J-1

| Compound | |
|---|---|
| (SEQ ID NO:) | Amino acid sequence |
| Natural antibacterial | Pro-Phe-Lys-Leu-Ser-Leu-His-Leu-NH₂ |
| polypeptide J-1 | |
| (SEQ ID NO: 16) | |
| 1 | Pro-Phe-Lys-Leu-Lys-Leu-His-Leu-NH₂ |
| 2 | Pro-Phe-Orn-Leu-Ser-Leu-Lys-Leu-NH₂ |
| 3 | Pro-Phe-Dab-Leu-Lys-Leu-Lys-Leu-NH₂ |
| 4 | Pro-Phe-Arg-Leu-Ser-Leu-His-Leu-NH₂ |
| 5 | Pro-Phe-Arg-Leu-Arg-Leu-His-Leu-NH₂ |
| 6 | Pro-Phe-Arg-Leu-Ser-Leu-Arg-Leu-NH₂ |
| 7 | Pro-Phe-Arg-Leu-Arg-Leu-Arg-Leu-NH₂ |
| 8 | Pro-Trp-Lys-Leu-Ser-Leu-His-Leu-NH₂ |
| 9 | Pro-Trp-Orn-Leu-Orn-Leu-His-Leu-NH₂ |
| 10 | Pro-Trp-Dab-Leu-Ser-Leu-Dab-Leu-NH₂ |
| 11 | Pro-Trp-Dap-Leu-Dap-Leu-Dap-Leu-NH₂ |
| 12 | Pro-Trp-Arg-Leu-Ser-Leu-His-Leu-NH₂ |
| 13 | Pro-Trp-Arg-Leu-Arg-Leu-His-Leu-NH₂ |
| 14 | Pro-Trp-Arg-Leu-Ser-Leu-Arg-Leu-NH₂ |
| 15 | Pro-Trp-Arg-Leu-Arg-Leu-Arg-Leu-NH₂ |

Another objective of the present invention is to provide a preparation method of a hydrogel.

Based on numerous experimental researches, the inventor of the present invention proves that the hydrogel exhibits antibacterial and hemostatic properties and may load various drugs or growth factors to realize the functional treatment with dressing, antibacterial and hemostatic functions in wound treatment, and maintain wet environment for the wound surface.

In order to achieve the above-mentioned objective, the hydrogel of the present invention is formed by polymerization reaction of the antibacterial polypeptide compound and a buffer solution.

The preparation method of the hydrogel of the present invention comprises the following steps:

S1, dissolving the antibacterial polypeptide compound in DMSO to obtain an antibacterial polypeptide compound solution for further use; and

S2, adding the antibacterial polypeptide compound solution to a buffer solution, and carrying out an ionic crosslinking polymerization reaction under ultrasonic or stirring conditions to obtain the hydrogel.

A solvent of the hydrogel of the present invention mostly comprises water, which is followed by DMSO, wherein the volume content of the DMSO is less than 5%.

Preferably, the preparation method provided by the present invention further comprises the following step:

S3, adding a drug and/or a growth factor to the buffer solution to obtain the hydrogel loaded with the drug or the growth factor.

Preferably, the drug of the present invention is an antibacterial drug or an anti-inflammatory drug, and the growth factor is a growth factor for promoting wound healing.

The buffer solution of the present invention is a carbonate solution, a sulfite solution, a phosphate buffered solution or the like, preferably the phosphate buffered solution; wherein, the phosphate buffered solution is prepared by dissolving Na₂HPO₄, KH₂PO₄, KCl and NaCl in deionized water in proportion; the antibacterial polypeptide compound and the phosphate buffered solution comprises the following components in molar ratio: the antibacterial polypeptide compound: Na₂HPO₄: KH₂PO₄: KCl:NaCl=(1-50):(1-10):(1-5):(1-5):(50-200); preferably, the antibacterial polypeptide compound:Na₂HPO₄:KH₂PO₄:KCl:NaCl=1-50:10:2:2.7:137.

Preferably, the phosphate buffered solution of the present invention further comprises adenosine diphosphate (ADP), and a molar ratio of the components of the phosphate buffered solution is Na₂HPO₄:KH₂PO₄:KCl:NaCl:ADP32 (1-10):(1-5):(1-5):(50-200):1; and preferably, the molar ratio of the ADP to Na₂HPO₄ is 1:10.

The reaction employed in the present invention may be a physical reaction or a chemical reaction, preferably, an ionic crosslinking polymerization reaction at a reaction temperature of 0°C.-60° C. for a reaction period of 1 min-120 mins.

A further objective of the present invention is to provide a use of a hydrogel in an anti-adhesion drug, the anti-adhesion drug comprises the hydrogel loaded with a drug or a growth factor and at least one pharmaceutically acceptable carrier and/or excipient.

The anti-adhesion drug of the present invention is in at least one dosage form of tablet, sugar-coated tablet, granule, drop, spray, rinse, mouthwash, ointment and paste applied on skin surface, and sterile solution for injection.

The drug of the present invention is an antibacterial drug or an anti-inflammatory drug, and the growth factor is a growth factor for promoting wound healing.

The hydrogel of the present invention is able to be directly used to wash, spray on, dress or cover the wound surface, or made into a convenient spray that is directly sprayed on the wound surface to form a protective film, thus instantly stopping bleeding, keeping the wound surface moist, creating hypoxic environment conducive to the growth and healing of epithelial cells, and accelerating wound healing. In addition, the antibacterial polypeptide in the hydrogel exhibits a quick-acting and durable broad-spectrum antibacterial effect during wound healing, and is decomposed into metabolizable amino acids after wound healing to avoid adhesion and residue.

In addition, the hydrogel of the present invention can also be used in proper manner and made into the appropriate dosage form according to location of disease or wound. For example, after debridement of wounds, contusions, abrasions, postoperative wounds, burns and scalds, and ulcers, the hydrogel of the present invention is used for spraying and substitution, or wet dressing and bandaging. The hydrogel of the present invention is used for spraying or wet dressing and bandaging on wounds after haemorrhoidectomy, anal abscess excision, anal fistulectomy, fissure excision, neostomy, fistulation, episiotomy and redundant circumcision. The hydrogel of the present invention is used for spraying or wet dressing on topical skin before and after radiotherapy. With respect to chronic non-healing wounds caused by diabetic foot, vasculitis and senile bedsore, the hydrogel of the present invention is used for spraying on the affected sites after debridement. In case of oral odor and postoperative care of oral surgery, the hydrogel of the present invention is made into mouthwash that directly contacts with the oral cavity and is spit out after gargling. The hydrogel of the present invention is used for spraying or wet dressing on surface of a wound of tinea, herpes, acne or the like. The hydrogel of the present invention is used for directly spraying or wet dressing on discomfort, itching, dry or peeling skin caused by skin irritation to improve the skin health.

The hydrogel of the present invention may also be loaded with various drugs or growth factors to realize functional treatment.

A further objective of the present invention is to provide a medical device, which comprises the antibacterial polypeptide compound or the hydrogel.

The antibacterial polypeptide compound or the hydrogel of the present invention is coated on at least one surface of the medical device to form a material.

The medical device of the present invention is in the form of any one selected from the group consisting of surgical dressing, fiber, mesh, powder, microsphere, sheet, sponge, foam, suture anchoring device, catheter, stent, surgical tack, plate and screw, drug delivery device, anti-adhesion barrier and tissue adhesive.

The fiber of the present invention is a fabric; the sheet is a membrane or a splint; and the suture anchoring device is a suture or a staple.

The antibacterial polypeptide compound of the present invention has high antibacterial activity, high enzymatic hydrolysis stability, high bioavailability and low cytotoxicity, and may form the hydrogel under specific conditions.

The hydrogel of the present invention does not adhere to wounds, has antibacterial activity and hemostatic properties, and is able to be used for loading and controlled-release of a drug, for example, an anti-inflammatory drug, an epidermal growth factor, a vascular growth factor or the like may be loaded; moreover, the hydrogel may accelerate healing of the wounds and reduce the formation of scar tissue fibers. In addition, the preparation method of the hydrogel of the present invention involves few processing steps and few types of raw materials and is convenient to perform. The medical device of the present invention comprises the antibacterial polypeptide compound or the hydrogel and realizes an efficient treatment in a more convenient way, and the medical device is able to be widely used in clinic.

### DESCRIPTION OF THE DRAWINGS

Embodiments of the present invention are described in detail below with reference to the attached drawings, wherein:

FIG. 1 shows the effects of compounds 2, 8, 9, 13 and 15 of the present invention on the survival rate of mice in the mouse septic peritonitis model.

FIG. 2 shows a histogram of evaluated hemolysis rates of the present invention.

FIG. 3 shows the effects of the compounds 2, 8, 9, 13 and 15 of the present invention and the natural antibacterial polypeptide J-1 on the proliferations of test cells.

FIG. 4 shows the effects of the compounds 2, 8, 9, 13 and 15 of the present invention and the natural antibacterial polypeptide J-1 on the permeability of inner and outer membranes of E. coli.

FIG. 5 shows survival rate curves of mice in different groups after intraperitoneal injection of the compounds 2, 8, 9, 13 and 15 (100 mg/kg, 300 mg/kg, 500 mg/kg, 700 mg/kg and 1000 mg/kg of body weight, respectively).

FIG. 6 shows hydrogel images and SEM images of the antibacterial polypeptide compound of the present invention.

FIG. 7 shows diagrams of the hemostatic properties of the hydrogel of the present invention in the mouse liver hemorrhage model.

### DETAILED DESCRIPTION OF THE INVENTION

The embodiments of the present invention will be described in detail hereafter in conjunction with the examples. However, those skilled in the art will appreciate that the following examples are only presented for purposes of illustration of the present invention and shall not be construed as restriction to the scope. The instruments used in the present invention include: a high performance liquid chromatograph (Delta 600) from Waters Corporation (US), a reversed-phase C18 column (XBridge BEH 130 Prep, 19 mm*250 mm) as a preparation column; a reversed-phase C8 column (4.6 mm*250 mm) as an analytical column; and a BrukerMaxis 4G mass spectrometer. The reagents employed include: Rink-amide-MBHA resin from Tianjin Nankai Hecheng Science & Technology Co., Ltd. (loading: 0.43 mol/g, 200-400 mesh); amino acids (N-A-Fmoc-protected), N-Hydroxybenzotriazole (HOBt), Oxy-benzotriazole-N,N,N′,N′-tetramethylurea-hexafluorophosphate (HBTU), diisopropylethylamine (DIEA) and triisopropylsilane (TIS) purchased from GL Biochem (Shanghai) Ltd.; dichloromethane, N,N-dimethylformamide (DMF), piperidine, methanol, trifluoroacetic acid (TFA), phenol and pyridine purchased from Tianjin Second Chemical Reagent Factory; and ninhydrin purchased from Shanghai Third Chemical Reagent Factory. Other instruments or reagents used, without specific manufacturers, are conventional products purchased from the market. Unless otherwise specified in the examples, the examples were carried out according to conventional conditions or the conditions recommended by manufacturers.

### 1. Preparation of An Antibacterial Polypeptide Compound

For the convenience of explanation, taking compounds 1-15 as examples, the preparation method of the antibacterial polypeptide compound of the present invention includes but is not limited to the following steps:

(1) Resin swelling and test: a certain amount of Rink-amide-MBHA resin (loading: 0.45 mmol/g) was added to a synthesizer, an appropriate amount of redistilled dichloromethane was added, soaked and stirred for 30 mins until the resin swelled completely, washed four times with redistilled DMF (2 mins per time), and then a ninhydrin test was conducted to check the cleanliness of the resin (the ninhydrin test reagents were pyridine, ninhydrin and phenol in a ratio of 2:1:1).

(2) Removal of an F-moc protecting group: washed four times with 20% redistilled piperidine+80% redistilled DMF (v/v) (3 mins per time) to remove the F-moc protecting group, washed four times with redistilled DMF (2 mins per time), and then a ninhydrin test was conducted to check the removal of the protecting group.

(3) Amino acid condensation reaction: triple amount of condensation agents HOBT, HBTU and amino acid (N-alpha-Fmoc-protected) were weighed and dissolve in redistilled DMF, sextuple amount of an initiator DIEA was added, mixed, and then added to the resulting resin without Fmoc in step (2) to start the condensation reaction. The mixture was stirred for one hour and the whole reaction process was protected by a flow of argon. The solvent was subjected to vacuum drying, washed four times with redistilled DMF (2 mins per time), and then a ninhydrin test was conducted to check for complete condensation of the amino acid. The above-mentioned F-moc protection reaction process and the condensation reaction process were performed per synthesis of one amino acid until the condensations of all sequences of the compounds 1-15 were completed. The amino acids involved include Fmoc-Pro-OH, Fmoc-Phe-OH, Fmoc-Lys (Boc)-OH, Fmoc-Orn (Boc)-OH, Fmoc-Dab (Boc)-OH, Fmoc-Dap (Boc)-OH, Fmoc-Arg (pbf)-OH, Fmoc-His (Trt)-OH, Fmoc-Leu-OH and Fmoc-Ser (tBu)-OH.

(4) Polypeptide chain cutting: after synthesis of the last amino acid, washed with 20% redistilled piperidine to remove the F-moc protecting group, subjected to a ninhydrin test, washed twice with redistilled dichloromethane and redistilled methanol in turn (3 mins per time), the synthesizer was sealed for three-hour vacuum drying until the resin was completely dry. After that, a cleavage reagent (TFA:Tris:H₂O=95:2.5:2.5) was added to react for three hours, during which the mixture was stirred for 1 min every 20 mins, and then dried with a rotary evaporator and stored in a refrigerator at −20° C.

(5) Extraction: the product was taken out of the refrigerator at −20° C., added with glacial ether, shaken vigorously and let stand for 10 mins. After complete stratification, the supernatant was extracted with double distilled water, followed by the subnatant. The extracted liquids were filled into 50 ml beakers and stored at −80° C. overnight, and then subjected to lyophilization to obtain crude polypeptide powder.

(6) Purification of polypeptide: \textcircled{1} Desalination: the crude polypeptide was desalinated using a sephadex G-25 column to remove small molecular weight impurities. A certain amount of the crude polypeptide (about 40 mg) was weighed and dissolved in 5% glacial acetic acid/aqueous solution (1 ml), loaded on the column and eluted with 5% glacial acetic acid/aqueous solution. The absorbance value was measured at 220 nm with a ultraviolet detector to collect liquids according to main absorbance peak, which were filled into 50 ml beakers and stored at −80° C. overnight, and then subjected to vacuum drying to obtain desalinated crude polypeptide powder. \textcircled{2} HPLC sample preparation: a certain amount of the desalinated crude polypeptide (about 40 mg) was weighed and dissolved in 20% acetonitrile/aqueous solution (5 ml), and loaded on a column after filtration. The column for purification was a μ-bondapak™ reversed-phase C18 column (19 mm×300 mm), and a gradient elution was carried out with 20%-80% acetonitrile/aqueous solution. The absorbance value was measured at 220 nm with the ultraviolet detector to collect liquids according to main absorbance peak, which were filled into 50 ml beakers and stored at −80° C. overnight, and then subjected to vacuum drying to obtain pure polypeptide powder. Amino acid sequences and mass spectra of the synthesized compounds 1-15 and the natural antibacterial polypeptide J-1 are shown in Table 2.

TABLE 2
Amino Acid Sequences and the Mass Spectra of the Compounds 1-15

| | | Theoretical | Actual |
|---|---|---|---|
| | | molecular | molecular |
| Compound | Amino acid sequence | weight | weight |
| Natural | Pro-Phe-Lys-Leu-Ser-Leu-His-Leu-NH₂ | 993.65 | 994.58 |
| antibacterial | | | |
| polypeptide | | | |
| J-1 | | | |
| Compound 1 | Pro-Phe-Lys-Leu-Lys-Leu-His-Leu-NH₂ | 993.65 | 994.58 |
| Compound 2 | Pro-Phe-Orn-Leu-Ser-Leu-Lys-Leu-NH₂ | 929.62 | 929.65 |
| Compound 3 | Pro-Phe-Dab-Leu-Lys-Leu-Lys-Leu-NH₂ | 956.68 | 957.62 |
| Compound 4 | Pro-Phe-Arg-Leu-Ser-Leu-His-Leu-NH₂ | 980.59 | 981.60 |
| Compound 5 | Pro-Phe-Arg-Leu-Arg-Leu-His-Leu-NH₂ | 1049.66 | 1050.59 |
| Compound 6 | Pro-Phe-Arg-Leu-Ser-Leu-Arg-Leu-NH₂ | 999.63 | 1000.63 |
| Compound 7 | Pro-Phe-Arg-Leu-Arg-Leu-Arg-Leu-NH₂ | 1068.70 | 1069.70 |
| Compound 8 | Pro-Trp-Lys-Leu-Ser-Leu-His-Leu-NH₂ | 991.60 | 992.60 |
| Compound 9 | Pro-Trp-Orn-Leu-Orn-Leu-His-Leu-NH₂ | 1008.66 | 1008.66 |
| Compound | Pro-Trp-Dab-Leu-Ser-Leu-Dab-Leu-NH₂ | 926.63 | 926.63 |
| 10 | | | |
| Compound | Pro-Trp-Dap-Leu-Dap-Leu-Dap-Leu-NH₂ | 897.70 | 898.70 |
| 11 | | | |
| Compound | Pro-Trp-Arg-Leu-Ser-Leu-His-Leu-NH₂ | 1019.60 | 1020.61 |
| 12 | | | |
| Compound | Pro-Trp-Arg-Leu-Arg-Leu-His-Leu-NH₂ | 1088.69 | 1089.68 |
| 13 | | | |
| Compound | Pro-Trp-Arg-Leu-Ser-Leu-Arg-Leu-NH₂ | 1038.65 | 1039.65 |
| 14 | | | |
| Compound | Pro-Trp-Arg-Leu-Arg-Leu-Arg-Leu-NH₂ | 1107.71 | 1108.72 |
| 15 | | | |

### 2. Determination of A Minimum Inhibitory Concentration of Antibacterial Polypeptide Compounds

With respect to the determination of antibacterial activity and antifungal activity of the antibacterial polypeptide compounds, the bacterial strains employed included: E. coli (ATCC 25922), E. coli (ATCC 43837), Pseudomonas aeruginosa (ATCC 27853), Klebsiella pneumoniae (ATCC 700603), Cronobacter sakazakii (ATCC 29544), Staphylococcus aureus (ATCC 29213), Bacillus subtilis (ATCC 23857) and Staphylococcus epidermidis (ATCC 12228); and the fungal strains employed included Candida albicans (ATCC 14053), Candida glabrata (ATCC 2001), Candida parapsilosis (ATCC 22019), Candida tropicalis (ATCC 750) and Candida krusei (ATCC 6258), all of which were standard strains from American Type Culture Collection. In addition, drug-resistant strains S-1a, S-2a, S-3a, S-4a, E-1b, E-2b, E-3b, E-4b, E-5b, P-1c, P-2c, P-3c, P-4c, P-5c, and clinically isolated fungal strains Candida glabrata 2-1 and Candida albicans 14-1, 14-2 and 14-3 were employed, all which were clinically isolated.

The minimum inhibitory concentration (MIC) of the compounds against the bacteria and the fungi were determined by a standard 2-fold serial dilution method recommended by the National Committee for Clinical Laboratory Standards (NCCLS). Simply, a proper amount of the cryopreserved bacterium or fungus was added to fresh MH/SD medium, and cultured on a shaker overnight at 180 rpm at 37° C. The obtained cells were seeded again and cultured on a shaker for 4-5 hours to obtain cells in logarithmic phase. The resulting cells were added to a 96-well plate (1×10⁵ CFU/ml, 100 μL/well), and then 100 μL of polypeptide with different concentrations (1-256 μM) twice the final concentrations was added to corresponding well according to the 2-fold serial dilution method. The fresh medium was used as a negative control, which was prepared in triplicate for each concentration. After dosing, the 96-well plate was placed in an incubator at constant 37° C. and humidity for 12 hours to observe the results. According to naked eye observations, the concentration of the compound in the first transparent well following the turbid wells was recorded as the MIC of the compound, namely, the MIC value of the compound to the bacterium or fungus. The MICs of the compounds 1-15 to the standard bacterial strains are shown in Table 3, and the results of the antibacterial activity of the compounds 1-15 to fungi are shown in Table 4. Taking antibacterial polypeptide J-1, ciprofloxacin and gentamicin as control groups, the antibacterial activities of the compounds 2, 8, 9, 13 and 15 against the drug-resistant strains are shown in Table 5.

As shown in Table 3 and Table 5, the compounds 1-15 exhibit excellent antibacterial activity and antifungal activity against both the test bacteria and fungi. Wherein, the antibacterial activity and the antifungal activity of the compound 7 and the compound 15 are 8-16 times higher than those of the natural antibacterial polypeptide J-1, and the antibacterial activity and the antifungal activity of other compounds of the present invention are 1-8 times higher than those of the antibacterial polypeptide J-1. It is worth mentioning that the compounds 1-15 exhibit excellent antibacterial activity against the clinically isolated drug-resistant strains.

TABLE 3
MICs of the Compounds 1-15 against the Standard Bacterial Strains

| | MIC (μM) | | | | | |
|---|---|---|---|---|---|---|
| | C. coli | P. aerugmosa | C. sakazakii | S. aureus | B. subtilis | S. epidermidis |
| Compound | ATCC25922 | ATCC27853 | ATCC29544 | ATCC29213 | ATCC23857 | ATCC12228 |
| J-1 | 32 | 32 | 64 | 128 | 32 | 64 |
| 1 | 32 | 32 | 32 | 128 | 32 | 32 |
| 2 | 16 | 8 | 32 | 64 | 16 | 16 |
| 3 | 32 | 32 | 32 | 128 | 16 | 16 |
| 4 | 32 | 32 | 32 | 128 | 16 | 32 |
| 5 | 16 | 32 | 32 | 128 | 16 | 32 |
| 6 | 8 | 32 | 16 | 64 | 16 | 16 |
| 7 | 8 | 8 | 32 | 64 | 8 | 16 |
| 8 | 16 | 16 | 16 | 64 | 16 | 32 |
| 9 | 8 | 8 | 16 | 64 | 16 | 32 |
| 10 | 16 | 32 | 8 | 32 | 16 | 16 |
| 11 | 16 | 64 | 16 | 32 | 16 | 16 |
| 12 | 16 | 32 | 16 | 32 | 16 | 8 |
| 13 | 8 | 4 | 8 | 32 | 8 | 16 |
| 14 | 8 | 8 | 8 | 32 | 8 | 16 |
| 15 | 4 | 8 | 8 | 8 | 4 | 8 |

TABLE 4
Antibacterial Activities of the Compounds 1-15 against Fungi

| | MIC (μM) | | | | |
|---|---|---|---|---|---|
| | C. glabrata | C. albicans | C. tropicalis | C. krusei | C. parapsilosis |
| Compound | ATCC22019 | ATCC14053 | ATCC750 | ATCC6258 | ATCC2001 |
| J-1 | 64 | 64 | 16 | 32 | 32 |
| 1 | 32 | 32 | 32 | 64 | 32 |
| 2 | 32 | 32 | 32 | 32 | 16 |
| 3 | 32 | 32 | 32 | 64 | 16 |
| 4 | 32 | 32 | 32 | 32 | 16 |
| 5 | 16 | 16 | 32 | 64 | 16 |
| 6 | 8 | 32 | 16 | 32 | 16 |
| 7 | 8 | 8 | 32 | 32 | 8 |
| 8 | 16 | 16 | 16 | 32 | 16 |
| 9 | 16 | 32 | 16 | 64 | 16 |
| 10 | 16 | 32 | 8 | 32 | 16 |
| 11 | 16 | 64 | 16 | 32 | 16 |
| 12 | 16 | 32 | 16 | 32 | 16 |
| 13 | 8 | 32 | 8 | 32 | 8 |
| 14 | 8 | 8 | 8 | 32 | 8 |
| 15 | 4 | 4 | 4 | 16 | 4 |

TABLE 5
Antibacterial Activities of the Compounds 2, 8, 9, 13 and 15 against Drug-resistant Strains

| | MIC (μM) | | | | | | | |
|---|---|---|---|---|---|---|---|---|
| | Natural | | | | | | | |
| | antibacterial | | | | | | | |
| | polypeptide | | | | | | | |
| Strain | J-1 | ciprofloxacin | gentamicin | Compound 2 | Compound 8 | Compound 9 | Compound 13 | Compound 15 |
| S-1a | 128 | — | — | 64 | 64 | 64 | 32 | 8 |
| S-2a | 128 | — | — | 64 | 64 | 64 | 32 | 8 |
| S-3a | 128 | — | — | 64 | 64 | 64 | 32 | 8 |
| S-4a | 128 | — | — | 64 | 64 | 64 | 32 | 8 |
| E-1b | 32 | 25 | 50 | 16 | 16 | 8 | 8 | 4 |
| E-2b | 32 | 25 | 50 | 16 | 16 | 8 | 8 | 4 |
| E-3b | 32 | 25 | 50 | 16 | 16 | 8 | 4 | 4 |
| E-4b | 32 | 25 | 50 | 8 | 16 | 8 | 8 | 4 |
| E-5b | 32 | 25 | 50 | 8 | 16 | 8 | 4 | 4 |
| P-1c | 32 | 50 | 50 | 8 | 16 | 8 | 8 | 8 |
| P-2c | 32 | 25 | 50 | 8 | 8 | 8 | 4 | 8 |
| P-3c | 32 | 25 | 25 | 8 | 8 | 8 | 4 | 8 |
| P-4c | 32 | 50 | 50 | 8 | 8 | 8 | 4 | 8 |
| P-5c | 32 | 50 | 50 | 8 | 8 | 8 | 4 | 8 |

a refers to clinical isolated drug-resistant strain MRSA;
b refers to clinical isolated drug-resistant strain ESBL;
c refers to clinical isolated drug-resistant strain PA.

### 3. In Vivo Antibacterial Evaluation of Antibacterial Polypeptide Compounds

The determination of the in vivo antibacterial activity of the antibacterial polypeptide compounds is to reflect the antibacterial activity in vivo by detecting the survival rate of mice infected with bacteria or fungi.

Taking compounds 2, 8, 9, 13 and 15 of the present invention as examples, the in vivo antibacterial effects of the antibacterial polypeptide compounds were analyzed. The mice used in this test were Kunming mice, which were placed in constant-temperature environment (22±1° C.). Simply, 36 Kunming mice (18-22 g of body weight) were randomly divided into 6 groups and intraperitoneally injected with 0.2 ml of 3×10⁸ CFU/mL E. coli (ATCC 25922) on Day 1 to establish mouse septic peritonitis model. The mice were administered intraperitoneally one hour after inoculation with the bacteria, and then the compounds 2, 8, 9, 13 and 15 were injected intraperitoneally at a dose of 20 mg/kg of body weight. Polymyxin B (20 mg/kg) was selected as a positive reference control, and a negative control group received intraperitoneal injection of sterile saline. After 7 days of observation, the survival rates of mice in administration groups and control groups were calculated to create survival curves.

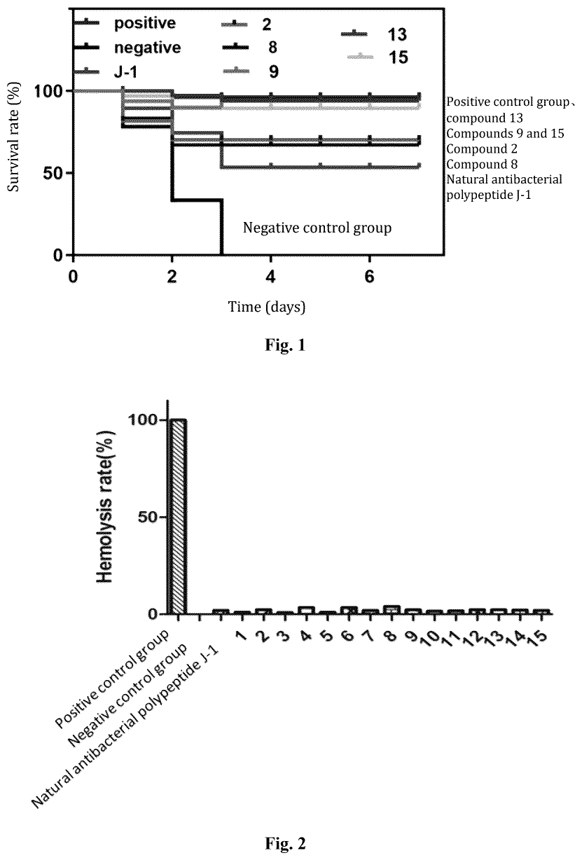

FIG. 1 shows a effects of the compounds 2, 8, 9, 13 and 15 of the present invention on the survival rate of mice in the mouse septic peritonitis model. As shown in FIG. 1, the mice received the compound 13 of the present invention and in the positive control group have the highest survival rate of 100%; the survival rates of the mice were similar between the compound 9 and the compound 15, above 90%; the survival rates of the mice with the compound 2 and the compound 8 were above 70%; followed by the survival rate of the mice with the natural antibacterial polypeptide J-1, about 50%; The lowest survival rate of mice was found in the negative control group, wherein all the mice died on Day 3. Compared with the negative control group and the natural antibacterial polypeptide J-1, the compounds 2, 8, 9, 13 and 15 of the present invention can effectively improved the survival rate of mice at the dose of 20 mg/kg. Therefore, it is proved that the antibacterial polypeptide compounds of the present invention exhibit high antibacterial activity in vivo.

### 4. Evaluation of Hemolytic Toxicity of Antibacterial Polypeptide Compounds

The hemolytic activity was determined by measuring a hemolytic concentration for mouse red blood cells with the antibacterial polypeptide compounds.

Taking compounds 1-15 of the present invention as examples, the hemolytic toxicity of the antibacterial polypeptide compounds was analyzed. The mouse red blood cells was diluted to a concentration of 8%, the obtained red blood cell suspension was added to a 96-well plate (100 μL per well), and then 100 μL of one compound was added to a corresponding well. After incubation for 1 hour, the supernatant of each well was added to another 96-well plate, and then the absorbance value of the sample supernatant was measured at 490 nm using a ELISA reader. With regard to the present invention, PBS and 2% Triton-X 100 were used as a negative control and a positive control, separately.

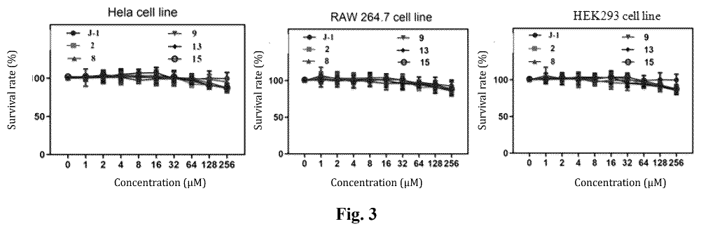

FIG. 2 shows a histogram of evaluated hemolysis rates of the present invention, wherein Triton X-100 was used as the positive control, with corresponding hemolysis rate of 100%; and PBS was used as the negative control, with corresponding hemolysis rate of 0. As shown in FIG. 2, the compounds 1-15 of the present invention exhibit almost no hemolytic activity on mouse red blood cells, and the hemolysis rates are less than 5%. Therefore, it is proved that the antibacterial polypeptide compounds of the present invention have low hemolytic activity.

### 5. Evaluation of Cytotoxicity of Antibacterial Polypeptide Compounds

For cytotoxicity determination, the MTT colorimetry was used to evaluate the effects of the compounds on the proliferation of human cervical cancer cell Hela, human embryonic renal epithelial cell HEK293 and mouse mononuclear macrophage RAW264.7.

Taking compounds 2, 8, 9, 13 and 15 of the present invention as examples, the cytotoxicity of the antibacterial polypeptide compounds was analyzed. Three cell lines were employed for the present invention, namely human cervical cancer cell Hela, human embryonic renal epithelial cell HEK293 and mouse mononuclear macrophage RAW264.7. Hela cells were cultured in RPMI 1640 medium with 10% newborn calf serum, HEK293 cells and RAW264.7 cells were cultured in DMEM medium with 10% fetal bovine serum. Simply, cells (1×10⁴ cells/well) were inoculated to a 96-well plate and incubated in an incubator set to 5% CO₂ at 37° C. After 24 hours, compounds of different concentrations were added and incubated in the 5% CO₂ incubator. After 24 hours, 20 μL of MTT solution with a concentration of 5 mg/mL was added to each well and incubated for 4 hours. After that, the medium was removed with a pipet, and 150 μL of DMSO added to dissolve the purple-blue formazan crystals. The solution was shaken in an oscillator for 5 mins before detecting the absorbance value at 570 nm using the ELISA reader, and then the survival rate was calculated with the formula: (OD dosing group/OD control group)×100% (n=3).

FIG. 3 shows the effects of the compounds 2, 8, 9, 13 and 15 of the present invention and the natural antibacterial polypeptide J-1 on the proliferations of test cells; wherein the graph on the left shows the effects of the compounds 2, 8, 9, 13 and 15 and the natural antibacterial polypeptide J-1 on the proliferation of Hela cell line; the graph in the middle shows the effects of the compounds 2, 8, 9, 13 and 15 on the proliferation of RAW 264.7 cell line; the graph on the right shows the effects of the compounds 2, 8, 9, 13 and 15 on the proliferation of HEK293 cell line; As shown in FIG. 3, the compounds 2, 8, 9, 13 and 15 exhibit no inhibitory activity on the three test cell lines. Therefore, it is proved that the antibacterial polypeptide compounds of the present invention have no cytotoxicity.

### 6. Evaluation of Permeability of E. coli Outer Membrane (OM) with Antibacterial Polypeptide Compounds

The destruction of bacterial cell OM was determined by using hydrophobic fluorescent probe NPN (1-N-phenylnaphthylamine) to determine the effect of the compounds on the OM permeability of E. coli (ATCC 25922).

Taking compounds 2, 8, 9, 13 and 15 of the present invention as examples, the OM permeability of E. coli with the presence of the antibacterial polypeptide compounds was analyzed. E. coli cells were washed twice with PBS solution and resuspended in 5 mM HEPES (pH 7.2), and then diluted to a concentration of 5*10+CFU/mL. The concentrations of the compounds ranged from 1×MIC to 8×MIC, an experimental group: 100 μL of cells+50 μL of Jelleine-1+50 μL of NPN (40 μM); and a control group: 100 μL of cells +50 μL of HEPES (5 mM)+50 μL of NPN (40 μM). The excitation wavelength and emission wavelength were set to 350 nm and 420 nm, separately, and the cells were detected once every minute using a multifunctional ELISA reader within a 15-min period of time.

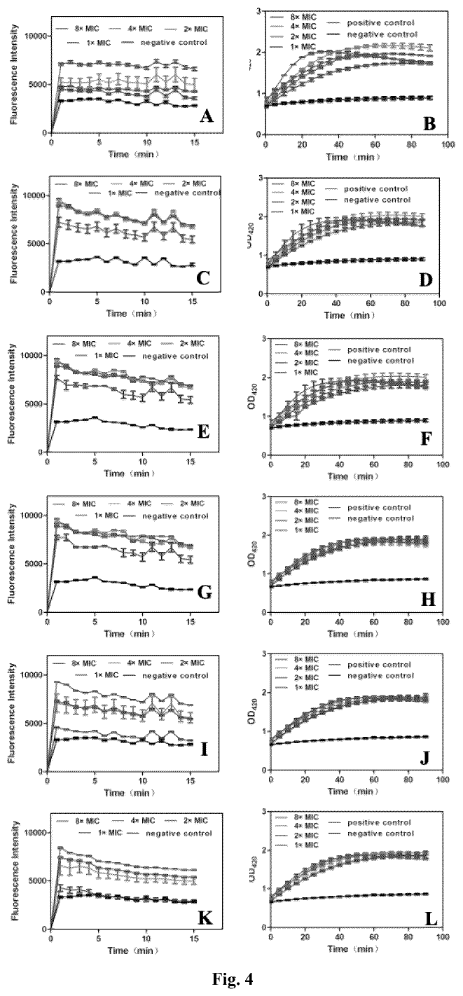

FIG. 4 shows the effects of the compounds 2, 8, 9, 13 and 15 of the present invention and the natural antibacterial polypeptide J-1 on the permeability of inner and outer membranes of E. coli; wherein, FIG. 4 (A, C, E, G, I, K) shows the effects of the natural antibacterial polypeptide J-1 and the compounds 2, 8, 9, 13 and 15 of different doses on the OM permeability of E. coli, wherein HEPES buffer solution was used as a negative control; it can be seen from the results in FIG. 4 (A, C, E, G, I, K) that the compounds 2, 8, 9, 13 and 15 and the antibacterial polypeptide J-1 may destroy the integrity of the E. coli OM in a dose-dependent manner.

### 7. Evaluation of Permeability of E. coli Inner Membrane (IM) with Antibacterial Polypeptide Compounds

The effect on the integrity of bacterial OM was determined by determining the release amount of β-galactosidase from E. coli ML-35 (ATCC 43837) with ONPG (o-nitrobenzene-β-D-galactoside), thereby detecting the effect of the compounds on the permeability of E. coli IM.

Taking compounds 2, 8, 9, 13 and 15 of the present invention as examples, the IM permeability of E. coli with the presence of the antibacterial polypeptide compounds was analyzed. E. coli ML-35 cells were washed twice with PBS solution and resuspended in 0.9% sodium chloride solution. The concentrations of the compounds ranged from 1×MIC to 8×MIC, an experimental group: 100 μL of cells+90 μL of Jelleine-1+10 μL of ONPG (30 mM); a positive control group: 100 μL of cells+90 μL of 1% Triton-X100+10 μL of ONPG (30 mM); and a negative control group: 100 μL of cells+90 μL of 0.5% sodium chloride solution+10 μL of ONPG (30 mM). The cells were detected at 420 nm once every 5 mins using a multifunctional ELISA reader within a 90-min period of time.

FIG. 4 shows the effects of the compounds 2, 8, 9, 13 and 15 of the present invention and the natural antibacterial polypeptide J-1 on the permeability of inner and outer membranes of E. coli. FIG. 4 (B, D, F, H, J, L) shows the effects of the antibacterial polypeptide J-1 and the compounds 2, 8, 9, 13 and 15 of different doses on the IM permeability of E. coli, wherein triton X-100 was used as a positive control, and normal saline was used as a negative control; it can be seen from the results in FIG. 4 (B, D, F, H, J, L) that the compounds 2, 8, 9, 13 and 15 and the natural antibacterial polypeptide J-1 are similar in destroying the integrity of the E. coli IM in a dose-dependent manner.

### 8. Evaluation of Acute Toxicity of Antibacterial Polypeptide Compounds

Acute toxicity of the compounds was determined by measuring the acute toxicity in mice with the compounds.

Taking compounds 2, 8, 9, 13 and 15 of the present invention as examples, the IM permeability of E. coli with the presence of the antibacterial polypeptide compounds was analyzed. The mice used in this test were Kunming mice, which were placed in constant-temperature environment (22±1° C.) and could freely obtain water and food. Simply, 132 healthy Kunming mice were randomly divided into 11 groups, with 12 mice in each group (half male and half female), wherein, 5 groups were intraperitoneally injected with 0.2 ml of the compounds of different doses (100-1000 mg/kg), and the control group was intraperitoneally injected with normal saline. In this experiment, melittin was used as a reference control, and the other 5 groups were intraperitoneally injected with melittin of different doses (10-32 mg/kg). The state of mice was observed and recorded after 2 hours of administration, and then observed twice a day for 14 days, and the survival rates of the mice were recorded.

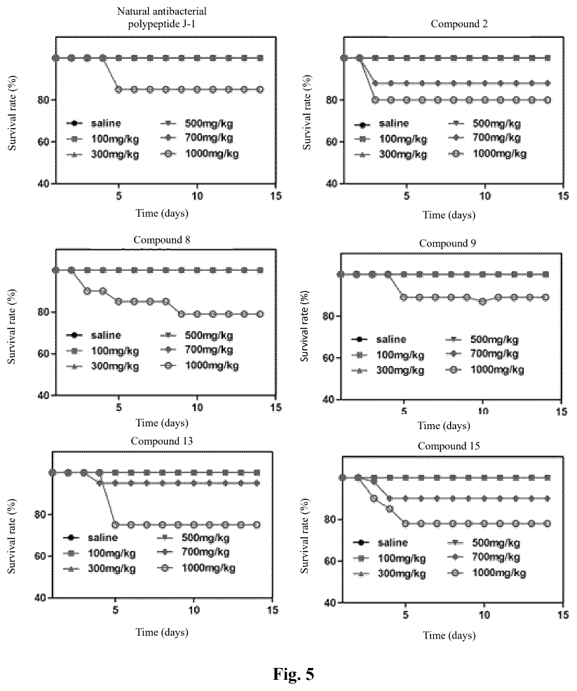

FIG. 5 shows the survival rate curves of mice in different groups after intraperitoneal injection of the compounds 2, 8, 9, 13 and 15 (100 mg/kg, 300 mg/kg, 500 mg/kg, 700 mg/kg and 1000 mg/kg of body weight, respectively). As shown in FIG. 5, 1-3 mice died in each of the highest dose (1000 mg/kg) groups of the compounds 2, 8, 9, 13 and 15, and there was no significant behavioral abnormality in the other groups. The LD₅₀ of melittin magainin was calculated as 20 mg/kg according to the Karber method, and the LD₅₀ of the compounds 2, 8, 9, 13 and 15 was more than 1000 mg/kg, so their acute toxicity was at least 50 times lower than that of melittin magainin. Therefore, it can be proven that the antibacterial polypeptide compounds of the present invention have low acute toxicity.

### 9. Preparation of A Hydrogel

The hydrogel of the present invention includes the following raw materials in molar ratio of: antibacterial polypeptide compound: 1-50 mM; Na₂HPO₄: 1-10 mM; KH₂PO₄: 1-5 mM; KCl: 1-5 mM; NaCl: 50-200 mM; and A solvent of the hydrogel mostly includes water, followed by DMSO, and the volume content of the DMSO is less than 5% (v/v).

Specifically, the preparation method of the hydrogel of the present invention includes the following step:

S1, the compound was dissolved in DMSO to prepare a polypeptide compound stock solution; and Na₂HPO₄, KH₂PO₄, KCL, and NaCl were dissolved in deionized water according to their above-mentioned concentrations for further use.

S2, the DMSO-dissolved antibacterial polypeptide J-1 solution was added to the above-mentioned saline solution to a final concentration of 1.5-40 mM, and uniformly stirred to obtain the hydrogel.

S3, in the preparation process of the hydrogel in S2, a drug or a growth factor was added to the saline solution to obtain the hydrogel controlled-release dressing loaded with the drug or the growth factor.

Taking the compound 8 of the present invention as an example, firstly, the compound 8 was dissolved in DMSO to prepare a high-concentration compound stock solution, which was added to a phosphate buffered solution (PBS, pH 6.0-9.0) and diluted to the final concentration of 10 mM, and then stirred for a proper time to obtain the hydrogel.

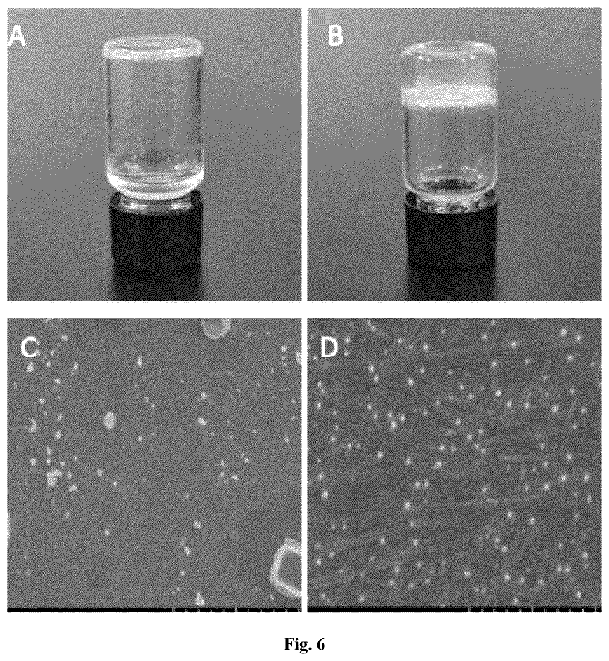

FIG. 6 shows hydrogel images and SEM images of the antibacterial polypeptide compound of the present invention; wherein, A shows the antibacterial polypeptide in liquid state; B shows the hydrogel image of the antibacterial polypeptide compound of the present invention; C shows the SEM image of the antibacterial polypeptide compound of the present invention dissolved in deionized water and dried at room temperature; and D shows the SEM image of the hydrogel of the present invention dried at room temperature.

### 10. Determination of Hemostatic Properties of A Hydrogel in A Mouse Liver Hemorrhage Model

The hemostatic properties were determined with the hydrogel prepared in the above-mentioned process. In the hemostatic property test with a hydrogel, only male Kunming mice (18 g-22 g of body weight) were used, which were raised at the temperature of 22° C.-24° C. and the relative humidity of 45%-55%, and fasted 12 hours before operation.

Liver hemorrhage modeling: In the experiment, mice were divided into two groups: a control group and a compound 8 hydrogel group, with 8 mice in each group. Each mouse was anesthetized at a dose of 40 mg/kg of body weight, positioned on a operating table with abdominal skin shaved, and the operative site was disinfected with iodophor. A longitudinal incision with a diameter of about 1.5 cm was made in the abdomen, and tissues were detached layer by layer to fully expose the right lobe of liver. A piece of filter paper weighed in advance was placed under the right lobe of liver, which was then punctured in the center with a 21 G needle. After that, 200 μL of the hydrogel was immediately applied on the wound (the control group was free from any treatment), and the site was photographed to record the liver hemorrhage process.

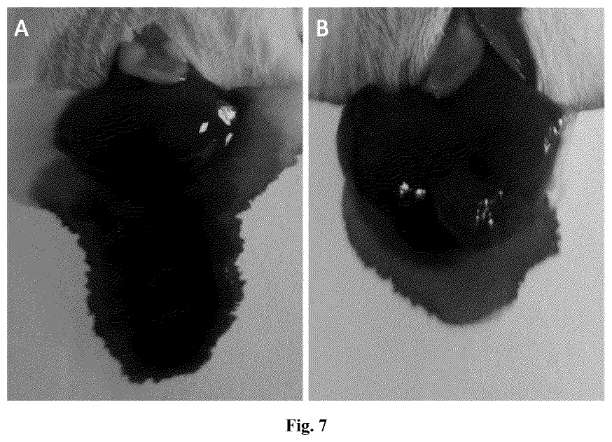

FIG. 7 shows diagrams of the hemostatic properties of the hydrogel of the present invention in the mouse liver hemorrhage model; wherein A indicates the normal saline control group; and B indicates the hydrogel group of the present invention. According to FIG. 7, compared with the control group, the hydrogel of the present invention remarkably inhibits liver hemorrhage. Therefore, it is proved that the hydrogel of the present invention has excellent hemostatic properties.

Although the present invention has been disclosed by the examples as above, it is not intended to limit the scope of the present invention. Any person skilled in the art with common knowledge may make little modifications and variations, without departing from the spirit and scope of the present invention. Therefore, as regards the scope of protection of the present invention, what is claimed herein shall prevail.

1. An antibacterial polypeptide compound, wherein the antibacterial polypeptide compound comprises a parent peptide represented by the following amino acid sequence:
Pro-Xaa2-Xaa3-Leu-Xaa5-Leu-Xaa7-Leu-NH₂
wherein, Xaa2=Phe, homo-Phe or Trp;
Xaa3=Lys, Aib, Orn, Dab, Dap or Arg;
Xaa5=Ser, Lys, Orn, Dab, Dap or Arg;
Xaa7=His, Lys, Orn, Dab, Dap or Arg;
and if Xaa2=Phe, Xaa3=Lys and Xaa5=Ser, Xaa7≠His.

2. The antibacterial polypeptide compound according to claim 1, wherein the antibacterial polypeptide compound comprises the following amino acid sequences:
compound 1:
Pro-Phe-Lys-Leu-Lys-Leu-His-Leu-NH₂
compound 2:
Pro-Phe-Orn-Leu-Ser-Leu-Lys-Leu-NH₂
compound 3:
Pro-Phe-Dab-Leu-Lys-Leu-Lys-Leu-NH₂
compound 4:
Pro-Phe-Arg-Leu-Ser-Leu-His-Leu-NH₂
compound 5:
Pro-Phe-Arg-Leu-Arg-Leu-His-Leu-NH₂
compound 6:
Pro-Phe-Arg-Leu-Ser-Leu-Arg-Leu-NH₂
compound 7:
Pro-Phe-Arg-Leu-Arg-Leu-Arg-Leu-NH₂
compound 8:
Pro-Trp-Lys-Leu-Ser-Leu-His-Leu-NH₂
compound 9:
Pro-Trp-Orn-Leu-Orn-Leu-His-Leu-NH₂
compound 10:
Pro-Trp-Dab-Leu-Ser-Leu-Dab-Leu-NH₂
compound 11:
Pro-Trp-Dap-Leu-Dap-Leu-Dap-Leu-NH₂
compound 12:
Pro-Trp-Arg-Leu-Ser-Leu-His-Leu-NH₂
compound 13:
Pro-Trp-Arg-Leu-Arg-Leu-His-Leu-NH₂
compound 14:
Pro-Trp-Arg-Leu-Ser-Leu-Arg-Leu-NH₂
compound 15:
Pro-Trp-Arg-Leu-Arg-Leu-Arg-Leu-NH₂.

3. A hydrogel, wherein the hydrogel is formed by polymerization reaction of the antibacterial polypeptide compound according to claim 1 and a buffer solution.

4. The hydrogel according to claim 3, wherein a preparation method of the hydrogel comprises the following steps:
S1, dissolving the antibacterial polypeptide compound in dimethyl sulfoxide (DMSO) to obtain an antibacterial polypeptide compound solution for further use; and
S2, adding the antibacterial polypeptide compound solution to a buffer solution, and carrying out an ionic crosslinking polymerization reaction under ultrasonic or stirring conditions to obtain the hydrogel.

5. The hydrogel according to claim 4, wherein the method further comprises the following step:
S3, adding a drug and/or a growth factor to the buffer solution to obtain the hydrogel loaded with the drug or the growth factor.

6. The hydrogel according to claim 5, wherein the drug is an antibacterial drug or an anti-inflammatory drug, and the growth factor is a growth factor for promoting wound healing.

7. The hydrogel according to claim 4, wherein the volume content of the DMSO is less than 5%.

8. The hydrogel according to claim 3, wherein the buffer solution is a phosphate buffered solution; and the antibacterial polypeptide compound and the phosphate buffered solution comprises the following components in molar ratio: the antibacterial polypeptide compound: Na₂HPO₄:KH₂PO₄:KCl:NaCl32 (1-50):(1-10):(1-5):(1-5):(50-200).

9. The hydrogel according to claim 8, wherein the phosphate buffered solution further comprises adenosine diphosphate (ADP), and a molar ratio of the ADP to Na₂HPO₄ is 1:(1-50).

10. The hydrogel according to claim 3, wherein the reaction is an ionic crosslinking polymerization reaction at a reaction temperature of 0° C.-60° C. for a reaction period of 1 min-120 mins.

11. The hydrogel according to claim 3, wherein the hydrogel is used in an anti-adhesion drug, and wherein the anti-adhesion drug comprises the hydrogel loaded with a drug and/or a growth factor and at least one pharmaceutically acceptable carrier and/or excipient.

12. The hydrogel according to claim 11, wherein the anti-adhesion drug is in at least one dosage form of tablet, sugar-coated tablet, granule, drop, spray, rinse, mouthwash, ointment and paste applied on skin surface, and sterile solution for injection.

13. The hydrogel according to claim 11, wherein the drug is an antibacterial drug or an anti-inflammatory drug, and the growth factor is a growth factor for promoting wound healing.

14. A medical device, wherein the medical device comprises the antibacterial polypeptide compound according to claim 1.

15. The medical device according to claim 14, wherein the antibacterial polypeptide compound is coated on at least one surface of the medical device to form a material.

16. The medical device according to claim 14, wherein the medical device is in the form of any one selected from the group consisting of surgical dressing, fiber, mesh, powder, microsphere, sheet, sponge, foam, suture anchoring device, catheter, stent, surgical tack, plate and screw, drug delivery device, anti-adhesion barrier and tissue adhesive.

17. The medical device according to claim 16, wherein the fiber is a fabric; the sheet is a membrane or a splint; and the suture anchoring device is a suture or a staple.
